(19) United States
(12) Patent Application Publication
(10) Pub. No.: US 2009/0117154 A1
(43) Pub. Date: May 7, 2009

(54) SYNTHETIC POLYVALENT CARBOHYDRATES AS COMPONENTS OF MICROBICIDES

(75) Inventors: Lai-Xi Wang, Ellicott City, MD (US); Jingsong Wang, Lutherville, MD (US); Timothy Fouts, Columbia, MD (US)

Correspondence Address: MOORE & VAN ALLEN PLLC, P.O. BOX 13706, Research Triangle Park, NC, 27709, US

(73) Assignee: UNIVERSITY OF MARYLAND BIOTECHNOLGY INSTITUTE, Baltimore, MD (US)

(21) Appl. No.: 12/066,963

(22) PCT Filed: Sep. 14, 2006
(86) PCT No.: PCT/US06/35856

§ 371 Date: Oct. 17, 2008

Related U.S. Application Data
(60) Provisional application No. 60/717,045, filed on Sep. 14, 2005.

(51) Int. Cl. A61K 39/21; C12N 7/00; A61K 31/715; G01N 33/566; C08L 77/00

(52) U.S. Cl. 424/208.1; 435/235.1; 514/54; 436/501; 525/54.2

(57) ABSTRACT

Inhibitors that block the DC-SIGN mediated transmission of the HIV-virus from mucosal infection sites to T-lymphocytes. In one embodiment, the inhibitors include at least one oligosaccharide chain attached to a scaffolding framework in which the number of the oligosaccharide chains attached to the scaffold can be 2, 3, 4 or more. In another embodiment, HIV-I viral infection is treated by administration of a composition including a therapeutically effective amount of an oligosaccharide cluster and/or oligosaccharide/protein cluster binding DC-SIGN, to inhibit DC-SIGN from binding to HIV envelope glycoprotein.

### CROSS-REFERENCE TO RELATED APPLICATION

The benefit of priority of U.S. Provisional patent Application No. 60/717,045 filed Sep. 14, 2005 in the name of lai-Xi, Jingsong Wang and Timothy F. Fouts, is hereby claimed under the provisions of 35 USC §119(e). The disclosure of said U.S. provisional application is hereby incorporated herein in its entirety, for all purposes.

### BACKGROUND OF THE INVENTION

1. Field of the Invention

The present invention relates to polyvalent carbohydrates, and more particularly, to synthesis of inhibitors that can block the DC-SIGN mediated transmission of the HIV-1 virus from mucosal infection sites to T-lymphocytes, and uses for topical microbicides.

2. Background of the Related Art

The specific interactions between HIV-1 surface glycans (carbohydrate ligands) and host cell surface lectins play an important role in HIV-1 attachment, infection, and transmission [1-9]. For example, DC-SIGN (dendritic cell-specific ICAM-3 grabbing nonintegrin), a C-type lectin expressed on dendritic cells, is largely responsible for the ability of dendritic cells to efficiently transmit HIV-1 to T-lymphocytes and to enhance HIV-1 infection in trans [1, 2]. DC-SIGN is specific for both high-mannose type oligosaccharides and fucose-containing Lewis type oligosaccharide antigens. These oligosaccharides are present on HIV-1 envelope glycoproteins. Therefore, blocking the specific carbohydrate-receptor interactions might reduce or prevent the HIV-1 attachment, infection, and transmission. There are some reports that polysaccharides such as mannan, and synthetic mannose-containing dendrimers can bind to DC-SIGN, thus implicating the potential to block the HIV carbohydrate-DC-SIGN interactions [2, 10]. Some bacterial proteins such as cyanovirin-N that binds specifically to oligomannose structure on gp120 are also effective to block the HIV-1 gp120 and DC-SIGN interactions and inhibit HIV-1 infection [11-14]. Therefore, these compounds are potentially useful as components for microbicide development. But a key point is the efficiency of the inhibition. To efficiently suppress the gp120 binding to DC-SIGN, the inhibitors must be at least as competitive as the gp120 itself. The higher the affinity of the inhibitor is to DC-SIGN, the more effective it would be as a component of microbicide. In addition, an important feature of the DC-SIGN mediated binding is multivalent interactions. Therefore, a multivalent ligand is expected to be more effective in preventing the interaction of gp120 and DC-SIGN, as exemplified by many examples in the biological system [15].

As stated above, DC-SIGN is specific for both high-mannose type oligosaccharides and fucose-containing Lewis oligosaccharides that are present on HIV-1 envelope glycoproteins. Thus, it would be advantageous to develop multivalent carbohydrate ligands, having an affinity for DC-SIGN, at least as great as gp120, thereby blocking the specific carbohydrate-receptor interactions that allow the HIV-1 attachment and subsequent infection.

### SUMMARY OF THE INVENTION

The present invention provides for methods of preparing novel multivalent carbohydrate ligands, and the use of the multivalent ligands as components of microbicides for preventing HIV-1 attachment, infection, and transmission.

In one aspect the present invention relates to a method for blocking DC-SIGN thereby inhibiting HIV carbohydrate-DC-SIGN interactions, the method comprising at least one oligosaccharide chain covalently attached to a scaffolding framework wherein number of the oligosaccharide chains attached to the scaffold could be 2, 3, 4, or more.

In another aspect, the present invention relates to at least one high-mannose oligosaccharide positioned on a scaffolding framework or molecule to form a high-mannose oligosaccharide cluster thereby generating a carbohydrate complex that binds with DC-SIGN.

In yet another aspect, the present invention relates to high-mannose oligosaccharide cluster comprising at least one high-mannose oligosaccharide assembled on a polyacrylamide (PAA) type polymer backbone.

In yet another aspect, the present invention relates to a vaccine comprising at least one oligosaccharide chain linked to a scaffolding framework wherein the number of the oligosaccharide chains attached to the scaffold could be 2, 3, 4, or more.

Another aspect of the present invention relates to methods for generating an oligosaccharide cluster comprising the steps of:
covalently linking or attaching at least one high-mannose oligosaccharide chain to a scaffold molecule to generate an oligosaccharide cluster that mimics an antigenic structure having affinity for DC-SIGN. The high-mannose oligosaccharide chains may be obtained from the digestion of soybean agglutinin or produced by chemical synthesis. High-mannose oligosaccharide chains can include any structural variant of Man₉ (containing 9 mannose residues), Man₈:, Man₇, Man₆, Man₅ or a combination thereof. Any combination of these high-mannose oligosaccharide chains may be attached to a scaffolding framework comprising PPA.

In another aspect, the present invention provides a process for detecting candidate compounds that potentially interact with DC-SIGN, the process comprising:
contacting DC-SIGN with an oligosaccharide cluster of the present invention to form a receptor ligand complex; and
contacting the receptor-ligand complex with a candidate compound and determine the level of displacement of the oligosaccharide cluster from complex with DC-SIGN.

In yet another aspect, the present invention relates to an assay method for determining a target molecule having binding affinity to the oligosaccharide cluster, the method comprising
coating a surface with the oligosaccharide cluster of the present invention
contacting the surface with a sample suspected of including the target molecule and determining presence of such target molecule.

In still another aspect, the present invention relates to a method of treating an HIV-1 virus infection, comprising:
administering to a patient a composition comprising a therapeutically effective amount of the oligosaccharide cluster and/or an oligosaccharide/protein cluster to bind DC-SIGN thereby inhibiting same from binding to HIV envelope glycoprotein.

The oligosaccharide cluster of the present invention may be administered alone or in a pharmaceutical composition as a vaccine in a therapeutically effective amount to inhibit binding of DC-SIGN with HIV and further treating HIV.

The compositions of the present invention may further comprise at least one antiviral agent. The antiviral agent may include any agent that inhibits entry into a cell or replication therein of an infectious virus, and specifically retroviruses, such as HIV viruses. The antiviral agents include, but not limited to nucleoside RT inhibitors, CCR5 inhibitors/antagonists, viral entry inhibitors and their functional analogs.

The pharmaceutical compositions may be administered alone or in combination with a therapeutically effective amount of at least one antiviral agent, including, but not limited to:

nucleoside RT inhibitors, such as Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC);
CCR5 inhibitors/antagonists, such as SCH-C, SCH-D, PRO 140, TAK 779, TAK-220, RANTES analogs, AK602, UK-427, 857, monoclonal antibodies;
viral entry inhibitors, such as Fuzeon (T-20), NB-2, NB-64, T-649, T-1249, SCH-C, SCH-D, PRO 140, TAK 779, TAK-220, RANTES analogs, AK602, UK-427, 857; and functional analogs or equivalents thereof.

These and other aspects of the present invention, will be apparent from the detailed description of the invention provided hereinafter

### BRIEF DESCRIPTION OF THE FIGURES

FIG. 1 shows a general scheme for preparing polyacrylamide (PAA)-based carbohydrates.

FIG. 2 shows structures of typical synthetic polyvalent carbohydrates

FIG. 3 shows the inhibitory effects of oligosaccharides and oligomannose-containing polymers on the binding of soluble DC-SIGN to immobilized HIV-1 Bal gp120

FIG. 4 shows the inhibitory effects of oligosaccharides and oligomannose-containing polymers on the binding of HIV-1 Bal virus to the DC-SIGN expressed BTIP-1 cells

### DETAILED DESCRIPTION OF THE INVENTION

### The Invention:

### Oligosaccharide Clusters

The present invention relates to high-mannose oligosaccharide clusters comprising at least one high-mannose oligosaccharide covalently attached or linked to a scaffold thereby forming a high-mannose oligosaccharide cluster. The high-mannose oligosaccharide is selected from the group consisting of Man₉ Man₈, Man₇, Man₆, Man₅ and any combination thereof. The high-mannose oligosaccharide can be isolated from soybean agglutinin or synthesized by techniques well known to one skilled in the art. Preferably, the scaffold framework comprises at least two high-mannose oligosaccharides, and more preferably, four Man₉ are covalently linked to the scaffolding.

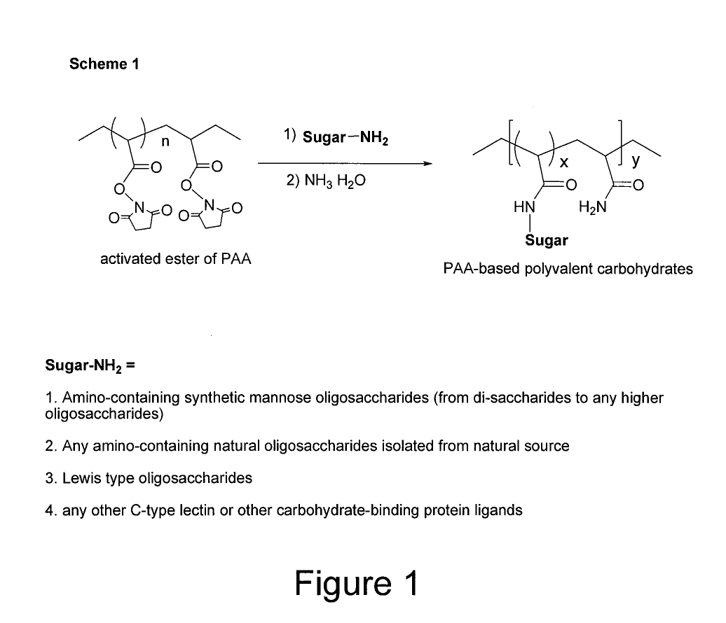

The present invention is based on polyvalent inhibitors prepared on polyacrylamide (PAA) mainly because PAA type polymer backbone is biologically compatible and non-toxic, and many PAA-based carbohydrates have been used for biomedical research and applications [16]. A general synthetic approach was demonstrated in Scheme 1, shown in FIG. 1. The synthesis uses a pre-activated PAA and let it react with oligosaccharides containing a free amino group. T his synthetic approach has been widely used for preparing PAA-based glycoconjugates [16]. To prepare efficient polyvalent ligands for DC-SIGN, the oligosaccharide portions will include all types of oligomannose in various glycosidic linkages such as α-1,2, α-1,3, and α-1,6-linkages. The oligosaccharides also include Lewis-type oligosaccharides and other oligosaccharides that are specific for C-type lectins. In addition, natural oligosaccharides such as the Man9GlcNAc2Asn can also be directly used for synthesizing the polyvalent compounds. The advantage of synthesis is that the extent of carbohydrate loading can be precisely controlled by change the ratio of the amino-oligosaccharides and the pre-activated PAA. In the second, the residual activated esters will be quenched by treatment with ammonia water to provide the PAA-based polyvalent carbohydrates. A simple procedure will allow a facile synthesis of the designed polyvalent carbohydrates.

The oligosaccharide clusters of the present invention may be administered as vaccines with various pharmaceutically acceptable carriers. Pharmaceutically acceptable carriers includes those approved for use in animals and humans and include diluents, adjuvants, excipients or any vehicle with which a compound, such as multivalent peptides and/or maleimide clusters, is administered. Pharmaceutically acceptable carriers include but are not limited to water, oils, saline, dextrose solutions, glycerol solutions, excipients such as starch, glucose, lactose, sucrose, gelatin, malt, rice, flour, chalk, silica gel, sodium stearate, glycerol monostearate, talc, sodium chloride, powdered non-fat milk, propylene glycol and ethanol. Pharmaceutical compositions may also include wetting or emulsifying agents, or pH buffering compounds.

### Pharmaceutical Compositions

The present invention provides for compositions comprising at least one oligosaccharide complex and optionally at least one antiviral agent wherein the two compounds can be administered, separately, simultaneously, concurrently or consecutively.

### Anti-Viral Compounds

In one aspect, the compositions and methods of the present invention may further comprise a therapeutically effective amount of at least one antiviral agent, including, but not limited to nucleoside RT inhibitors, CCR5 inhibitors/antagonists, viral entry inhibitors and functional analogs thereof.

Preferably, the antiviral agent comprises nucleoside RT inhibitors, such as Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC);

CCR5 inhibitors/antagonists, such as SCH-C, SCH-D, PRO 140, TAK 779, TAK-220, RANTES analogs, AK602, UK-427, 857, monoclonal antibodies;
viral entry inhibitors, such as Fuzeon (T-20), NB-2, NB-64, T-649, T-1249, SCH-C, SCH-D, PRO 140, TAK 779, TAK-220, RANTES analogs, AK602, UK-427, 857; and functional analogs thereof.

The present invention also contemplates pharmaceutical formulations, both for veterinary and for human medical use, which comprise as the active agent one or more compound(s) of the invention.

The carbohydrate complex according to the present invention, may be administered for therapy by any suitable route including oral, rectal, nasal, topical (including transdermal, buccal and sublingual), vaginal and parenteral (including subcutaneous, intramuscular, intravenous and intradermal). It will be appreciated that the preferred route will vary with the condition and age of the recipient, the nature of the infection and the chosen active ingredient.

In pharmaceutical formulations, the active agent preferably is utilized together with one or more pharmaceutically acceptable carrier(s) therefor and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired daily dose.

When the active agent is utilized in a formulation comprising a liquid solution, the formulation advantageously may be administered parenterally. When the active agent is employed in a liquid suspension formulation or as a powder in a biocompatible carrier formulation, the formulation may be advantageously administered orally, rectally, or bronchially.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

The formulations comprising the active agent of the present invention may conveniently be presented in unit dosage forms and may be prepared by any of the methods well known in the art of pharmacy. Such methods generally include the step of bringing the active compound(s) into association with a carrier that constitutes one or more accessory ingredients. Typically, the formulations are prepared by uniformly and intimately bringing the active compound(s) into association with a liquid carrier, a finely divided solid carrier, or both, and then, if necessary, shaping the product into dosage forms of the desired formulation.

Formulations suitable for parenteral administration conveniently comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Nasal spray formulations comprise purified aqueous solutions of the active compounds with preservative agents and isotonic agents. Such formulations are preferably adjusted to a pH and isotonic state compatible with the nasal mucous membranes.

Formulations for rectal administration may be presented as a suppository with a suitable carrier such as cocoa butter, hydrogenated fats, or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media, such as mineral oil, petroleum, polyhydroxy alcohols, or other bases used for topical pharmaceutical formulations.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

Formulations for rectal administration may be presented as a suppository with a suitable base comprising, for example, cocoa butter or a salicylate.

A preferred route of administration may be by application to vaginal mucosal surfaces. Formulations suitable for vaginal administration may be presented as pessaries, tampons, creams, gels, pastes, foams or spray formulations containing, in addition to the one or more of the compounds of the present invention, such carriers as are known in the art to be appropriate.

Further, the carbohydrate complexes of the present invention may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres that offer the possibility of local noninvasive delivery of drugs over an extended period of time. The administered therapeutic is slowly released from these microspheres and taken up by surrounding tissue cells (e.g. endothelial cells).

In addition to the aforementioned ingredients, formulations of this invention may further include one or more accessory ingredient(s) selected from diluents, buffers, flavoring agents, binders, disintegrants, surface active agents, thickeners, lubricants, preservatives (including antioxidants), and the like.

The compositions and methods of the present invention can be used to treat or reduce effects of HIV viral infection. At least oligosaccharide complex of the present invention may be administered for the treatment of HIV either as single therapeutic agents or when used in combination with antiretroviral drugs.

A composition of the present invention is typically administered parenterally in dosage unit formulations containing standard, well-known nontoxic physiologically acceptable carriers, adjuvants, and vehicles as desired. The term parenteral as used herein includes intravenous, intramuscular, intraarterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables. Preferred carriers include neutral saline solutions buffered with phosphate, lactate, Tris, and the like.

The compositions of the invention are administered in substantially non-toxic dosage concentrations sufficient to ensure the release of a sufficient dosage unit of the present complexes into the patient to provide the desired inhibition of the HIV virus. The actual dosage administered will be determined by physical and physiological factors such as age, body weight, severity of condition, and/or clinical history of the patient. The active ingredients are ideally administered to achieve in vivo plasma concentrations of an antiviral agent of about 0.01 uM to about 100 uM, more preferably about 0.1 to 10 uM, and most preferably about 1-5 uM, and of a oligosaccharide complex or mannose oligosaccharide/protein complex of about 1 u.M-25 uM, more preferably about 2-20 uM, and most preferably about 5-10 uM. It will be understood, however, that dosage levels that deviate from the ranges provided may also be suitable in the treatment of a given viral infection.

Therapeutic efficacy of the oligosaccharide complexes can be determined by standard pharmaceutical procedures in cell cultures or experimental animals, e.g., for determining The LD₅₀ (The Dose Lethal To 50% Of The Population) and The ED₅₀ (the dose therapeutically effective in 50% of the population). The dose ratio between toxic and therapeutic effects is the therapeutic index and it can be expressed as the ratio LD₅₀/ED₅₀. Compounds, which exhibit large therapeutic indexes, are preferred. The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Further, the therapeutic compositions according to the present invention may be employed in combination with other-therapeutic agents for the treatment of viral infections or conditions. Examples of such additional therapeutic agents include agents that are effective for the treatment of viral infections or associated conditions such as immunomodulatory agents such as thymosin, ribonucleotide reductase inhibitors such as 2-acetylpyridine 5-[(2-chloroanilino) thiocarbonyl) thiocarbonohydrazone, interferons such as alpha-interferon, 1-beta-D-arabinofuranosyl-5-(1-propynyl)uracil, 3′-azido-3′-deoxythymidine, ribavirin and phosphonoformic acid.

### EXPERIMENTAL PROCEDURES

### Synthetic Examples

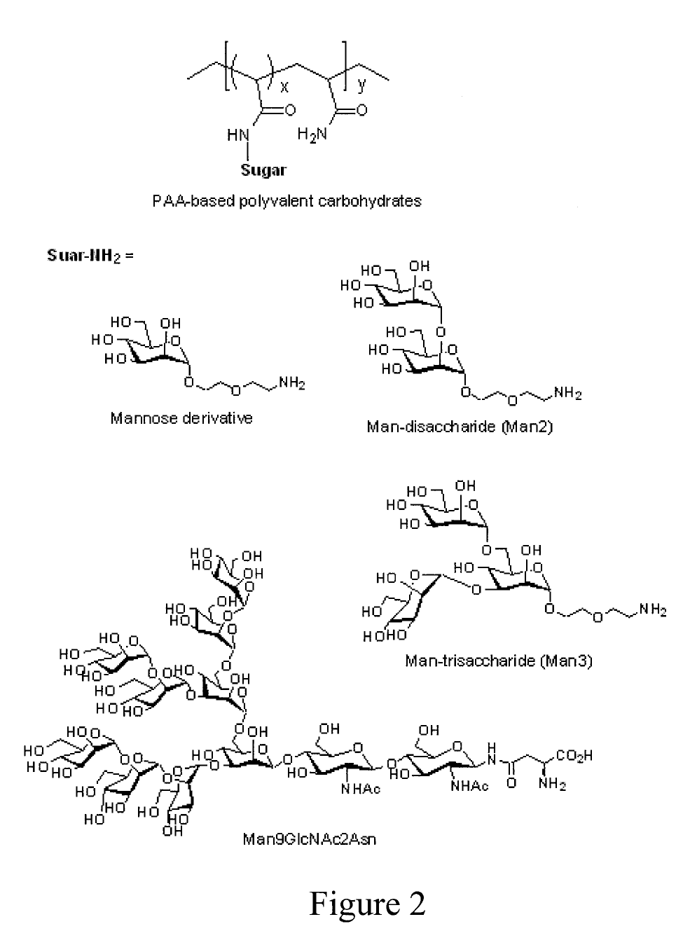

PAA-based, polyvalent carbohydrates containing multiple mannose, mannose disaccharides, mannose trisaccharides, and mannose oligosaccharides were prepared. The structures of polyvalent ligands are shown in FIG. 2. The NH2-containing mannose di- and tri-saccharides were chemically synthesized, and the Man9GlcNAc2Asn was prepared from soybean flour [17]. The approach can be extended to the preparation of other polyvalent carbohydrates such as those containing Lewis oligosaccharides and other carbohydrate ligands for various cell-surface lectins.

Inhibition of the DC-SIGN and HIV-1 gp120 interactions and the inhibition of the HIV-1 virus attachment to DC-SIGN expressing cells

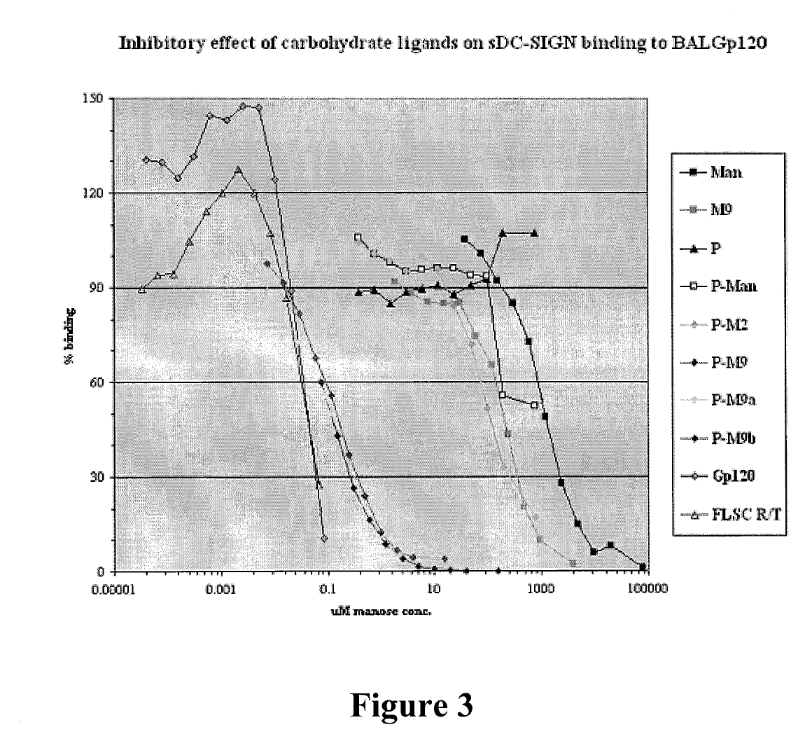

Preliminary assays were conducted on the inhibition of the synthetic polyvalent compounds against the binding between HIV-1 gp120 and DC-SIGN. Specifically, plates were coated with 5 ug/mL D7324 anti-gp120 capture antibody, in 1×PBS O/N at RT. Blocking buffer was 5% BSA in 1×TBST+1 mM CaCL₂, assay/dilution buffer was 2% BSA in 1×TBST+1 mM CaCL₂ and wash buffer was 1×TBST+1 mM CaCL₂ (4 washes, 200 uL per well). Gp120 (CHOK1, uQuant) was captured on the D7324 plate at saturation, 500 ng/mL in assay buffer for 2 hour at RT. sDC-SIGN supernatant (Nov. 6, 2004) was supplemented with 1 mM CaCL₂ and pre-complexed with 3 ug/mL anti-DC-SIGN mAb (120507, Sigma) for 1 hour at RT. Mannose products were titered 1:2 in assay buffer (Starts 1:4 in column 2), sDC-SIGN/mAb complexes were added to the titered mannose at about 1:5 (155 uL mannose+45 uL sDC-SIGN/mAb) (no sDC-SIGN in column 12), sDC-SIGN/mAb complexes mixed with mannose compounds were allowed to bind to the Gp120 plate for 1 hour at RT. Any bound sDC-SIGN/mAb complexes were detected with goat anti-mouse-IgG-HRP (Axcell) 1:2000 in assay buffer, absorbed with 2% sheep serum. The results were shown in FIG. 3. It was found that the polyvalent ligands containing the higher oligomannose were much more potent inhibitors than the polyvalent ligands containing mannose or mannose disaccharides. It was found that the Man9 is much better than the disaccharide and monosaccharide in inhibition, while the polyvalent Man9 is over 500-fold better than the Man9 itself in inhibiting the binding of DC-SIGN to gp120. In addition, it is important to point out that the Man9-containing PAA-based polyvalent ligands showed similar potency as HIV-1 envelope glycoprotein gp120 in inhibiting the binding of soluble DC-SIGN to immobilized gp120 (See FIG. 3).

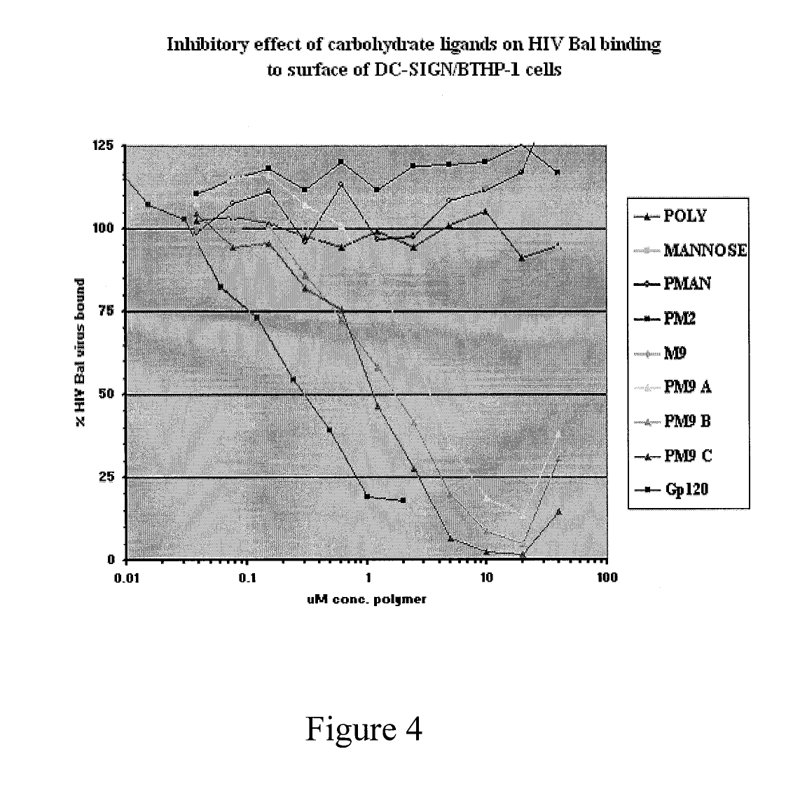

A further study involved the inhibitory effects of the synthetic polyvalent ligands on the binding of HIV-1 virus to cells expressing DC-SIGN, which mimics the DC-SIGN mediated HIV-1 transmission and infection. Specifically, an assay was conducted using BTHP-1 cells wherein DC-SIGN/BTHP-1 cells were plated at 1×10̂5/well in sterile non-treated v-bottom 96 well plates in complete RPMI media supplemented with 10% FBS, 1% L-glut and Pen-Strep. Mannose compounds were diluted in RPMI to 60 uM, and titered across the plate 1:3. Gp120 was diluted to 0.5 ug/mL, 2 uM, and titered 1:3. Cells were pelleted and resuspended in the diluted mannose compounds/Gp120 and incubated for 1 hour. Then the cells plus compounds were infected with HIV-1 BaL virus, 3500 TCID 50/mL final titer and incubated at 37 C for 3 hours. Cells were pelleted and unbound virus was removed from the cells by washing the cell pellet 5× with 200 uL RPMI. Final cell pellet was resuspended in 180 uL RPMI to which 20 uL 10× lysis buffer with TritonX-100 was added and allowed lysis to occur for 15 minutes at RT. Samples were then submitted for analysis by p24 ELISA. It was observed that the polyvalent Man9 showed potent inhibitory effect (see FIG. 4). The potency of the Man9-containing polymer in inhibition is approaching the soluble HIV-1 gp120.

### REFERENCES

The references discussed herein are hereby incorporated by reference herein for all purposes.
[1] Geijtenbeek, T. B., Kwon, D. S., Torensma, R., van Vliet, S. J., van Duijnhoven, G. C., Middel, J., Cornelissen, I. L., Nottet, H. S., KewalRamani, V. N., Littman, D. R., Figdor, C. G., and van Kooyk, Y. 2000. DC-SIGN, a dendritic cell-specific HIV-1-binding protein that enhances trans-infection of T cells. Cell 100:587-597.
[2] Geijtenbeek, T. B., and van Kooyk, Y. 2003. DC-SIGN: a novel HIV receptor on DCs that mediates HIV-1 transmission. Curr Top Microbiol Immunol 276:31-54.
[3] Ji, X., Gewurz, H., and Spear, G. T. 2005. Mannose binding lectin (MBL) and HIV. Mol Immunol 42:145-152.
[4] Hong, P. W., Flummerfelt, K. B., de Parseval, A., Gurney, K., Elder, J. H., and Lee, B. 2002. Human immunodeficiency virus envelope (gp120) binding to DC-SIGN and primary dendritic cells is carbohydrate dependent but does not involve 2G12 or cyanovirin binding sites: implications for structural analyses of gp120-DC-SIGN binding. J Virol 76:12855-12865.
[5] Dwek, R. A., Butters, T. D., Platt, F. M., and Zitzmann, N. 2002. Targeting glycosylation as a therapeutic approach. Nat Rev Drug Discov 1:65-75.
[6] Mitchell, D. A., Fadden, A. J., and Drickamer, K. 2001. A novel mechanism of carbohydrate recognition by the C-type lectins DC-SIGN and DC-SIGNR. Subunit organization and binding to multivalent ligands. J Biol Chem 276:28939-28945.
[7] Pohlmann, S., Baribaud, F., Lee, B., Leslie, G. J., Sanchez, M. D., Hiebenthal-Millow, K., Munch, J., Kirchhoff, F., and Doms, R. W. 2001. DC-SIGN interactions with human immunodeficiency virus type 1 and 2 and simian immunodeficiency virus. J Virol 75:4664-4672.
[8] Feinberg, H., Mitchell, D. A., Drickamer, K., and Weis, W. I. 2001. Structural basis for selective recognition of oligosaccharides by DC-SIGN and DC-SIGNR. Science 294:2163-2166.
[9] Saifuddin, M., Hart, M. L., Gewurz, H., Zhang, Y., and Spear, G. T. 2000. Interaction of mannose-binding lectin with primary isolates of human immunodeficiency virus type 1. J Gen Virol 81:949-955.
[10] Frison, N., Taylor, M. E., Soilleux, E., Bousser, M. T., Mayer, R., Monsigny, M., Drickamer, K., and Roche, A. C. 2003. Oligolysine-based oligosaccharide clusters: selective recognition and endocytosis by the mannose receptor and dendritic cell-specific intercellular adhesion molecule 3 (ICAM-3)-grabbing nonintegrin. J Biol Chem 278:23922-23929.
[11] Bolmstedt, A. J., O'Keefe, B. R., Shenoy, S. R., McMahon, J. B., and Boyd, M. R. 2001. Cyanovirin-N defines a new class of antiviral agent targeting N-linked, high-mannose glycans in an oligosaccharide-specific manner. Mol Pharmacol 59:949-954.
[12] Mori, T., and Boyd, M. R. 2001. Cyanovirin-N, a potent human immunodeficiency virus-inactivating protein, blocks both CD4-dependent and CD4-independent binding of soluble gp120 (sgp120) to target cells, inhibits sCD4-induced binding of sgp120 to cell-associated CXCR4, and dissociates bound sgp120 from target cells. Antimicrob Agents Chemother 45:664-672.
[13] Shenoy, S. R., Barrientos, L. G., Ratner, D. M., O'Keefe, B. R., Seeberger, P. H., Gronenborn, A. M., and Boyd, M. R. 2002. Multisite and multivalent binding between cyanovirin-N and branched oligomannosides: calorimetric and NMR characterization. Chem Biol 9:1109-1118.
[14] Turpin, J. A. 2002. Considerations and development of topical microbicides to inhibit the sexual transmission of HIV. Expert Opin Investig Drugs 11: 1077-1097.
[15] Mammen, M., Choi, S. K., and Whitesides, G. M. 1998. Polyvalent interactions in biological systems: Implications for design and use of multivalent ligands and inhibitors. Angew. Chem. Int. Ed. 37:2754-2794.
[16] Bovin, N. V. 1998. Polyacrylamide-based glycoconjugates as tools in glycobiology. Glycoconj J 15:431-446.
[17] Wang, L. X., Ni, J., Singh, S., and Li, H. 2004. Binding of high-mannose-type oligosaccharides and synthetic oligomannose clusters to human antibody 2G12: implications for HIV-1 vaccine design. Chem Biol 11: 127-134.

That which is claimed is:

1. A method for blocking DC-SIGN thereby inhibiting HIV carbohydrate-DC-SIGN interactions, the method comprising at least one oligosaccharide chain attached to a scaffolding framework wherein number of the oligosaccharide chains attached to the scaffold could be 2, 3, 4, or more.

2. The method according to claim 1, wherein the scaffolding framework is a polyacrylamide polymer backbone.

3. The method according to claim 1, wherein the oligosaccharide chain comprises any structural variant of Man₉ (containing 9 mannose residues), Man₈:, Man₇, Man₆, Man₅ or a combination thereof.

4. The method according to claim 1, wherein the oligosaccharide chain is a oligomannose in various glycosidic linkages such as α-1,2, α-1,3, and α-1,6-linkages, Lewis-type oligosaccharides and natural oligosaccharides such as the Man9GlcNAc2Asn.

5. A carbohydrate complex that binds with DC-SIGN comprising at least one oligosaccharide chain positioned on a scaffolding framework or molecule.

6. The carbohydrate complex according to claim 5, wherein the at least one oligosaccharide chain is assembled on a polyacrylamide (PAA) type polymer backbone.

7. The carbohydrate complex according to claim 6, wherein the oligosaccharide chain comprises any structural variant of Man₉ (containing 9 mannose residues), Man₈:, Man₇, Man₆, Man₅ or a combination thereof.

8. The carbohydrate complex according to claim 6, wherein the oligosaccharide chain is a oligomannose in various glycosidic linkages such as α-1,2, α-1,3, and α-1,6-linkages, Lewis-type oligosaccharides and natural oligosaccharides such as the Man9GlcNAc2Asn.

9. A vaccine comprising at least one oligosaccharide chain linked to a scaffolding framework wherein the number of the oligosaccharide chains attached to the scaffold could be 2, 3, 4, or more.

10. A method of treating an HIV-1 virus infection, comprising:
administering to a patient a composition comprising a therapeutically effective amount of the oligosaccharide cluster and/or an oligosaccharide/protein cluster to bind DC-SIGN thereby inhibiting same from binding to HIV envelope glycoprotein.

11. The method according to claim 10, wherein the composition further comprises at least one antiviral agent.

12. The method according to claim 10, wherein the antiviral agent may include any agent that inhibits entry into a cell or replication therein of an infectious virus.

13. An assay method for determining a target molecule having binding affinity to an oligosaccharide cluster, the method comprising
coating a surface with the oligosaccharide cluster of the present invention
contacting the surface with a sample suspected of including the target molecule and determining presence of such target molecule.
